(19) United States
(12) Patent Application Publication
(10) Pub. No.: US 2002/0160055 A1
(43) Pub. Date: Oct. 31, 2002

(54) Methods for treating human immunodeficiency virus infections with gallium compositions

(76) Inventors: Jack Stapleton, Iowa City, IA (US); Bradley Britigan, Iowa City, IA (US); Larry Schlesigner, Iowa City, IA (US)

Correspondence Address: FULBRIGHT & JAWORSKI L.L.P., SUITE 2400, 600 CONGRESS AVENUE, AUSTIN, TX, 78701, US

(21) Appl. No.: 09/954,975

(22) Filed: Sep. 18, 2001

Related U.S. Application Data
(60) Provisional application No. 60/233,353, filed on Sep. 18, 2000.

(51) Int. Cl. A61K 31/555; A61K 33/24

(52) U.S. Cl. 424/617; 514/184

(57) ABSTRACT

The present invention provides for the use of gallium compositions for the treatment of HIV. In particular, combination therapies are provided in which gallium is used to potentiate the effect of other antiviral drugs, such as nucleoside analogs.

This application claims benefit of the filing date of U.S. Provisional Patent Application Serial No. 60/233,353 filed on Sep. 18, 2000. The entire text of the above-referenced disclosure is specifically incorporated by reference herein without disclaimer.

The government owns rights in the present invention pursuant to grant number NIAA 1R21AA0906, 1R01AI0040 and RO3 AI43870 from the National Institutes of Health.

### BACKGROUND OF THE INVENTION

1. Field of the Invention

The present invention relates generally to the fields of virology and molecular biology. More particularly, it concerns the use of gallium compositions for the treatment of subject infected with human immunodeficiency virus.

2. Description of Related Art

The Human Immunodeficiency Virus (HIV) epidemic continues to grow at a rapid rate, and the clinical manifestations associated with this viral infection present increasingly more complex medical and socioeconomic problems. Acute HIV infection leads to a period of rapid viral replication, followed by viremia that results in infection of 1% or more of circulating T lymphocytes, the primary target of the virus. Viremia is transient, however, because the cells infected with HIV are removed from circulation by an effective host immune response that results in a 10-to 100-fold decrease in the HIV-infected T cells. Subsequently, the host's ability to protect itself immunologically from various challenges degrades, giving rise to acquired immunodeficiency syndrome, or AIDS.

No prevention or cure has yet been found for HIV infection and AIDS. Current treatments for HIV infection attempt to retard the progress of the disease or relieve its symptoms. Treatment in use today include certain dideoxynucleotides such as azidothymidine (AZT or zidovudine, Burroughs Wellcome), dideoxyinosine (ddI, Bristol-Myers Squibb) or dideoxycytidine (ddC, Hoffman-LaRoche). However, these agents can be toxic, and their applicability is limited because of the appearance in some patients of onerous, and sometimes lethal, side effects. These side effects include myelosuppression, peripheral neuropathy, and pancreatitis. In some patients, AZT loses effectiveness after prolonged use. While other drugs have been proposed for treatment of HIV infection, including the recent introduction of several HIV protease inhibitors, none have yet been demonstrated to be completely effective. Therefore, there remains a need in the art to develop additional therapeutic agents to prevent and treat HIV infection.

The conversion of the four ribonucleotides (ATP, GTP, CTP and TTP) to deoxyribonucleotides (dNTP's) is a rate-limiting step for DNA synthesis, and is catalyzed by ribonucleotide reductase (Rr). Inhibition of Rr inhibits DNA synthesis by depleting dNTP precursors (Thelander & Reichard, 1979). Since HIV-1 proviral DNA synthesis is significantly faster than host cell DNA synthesis, selective inhibition of proviral DNA synthesis may result from Rr inhibition. In addition, since dideoxynucleotides compete with endogenous dNTP's for inhibition of HIV-1 reverse transcriptase, depleting dNTP pools with Rr inhibitors may potentiate the activity of these therapeutic agents. Ribonucleotide reductase is the site of action of hydroxyurea (HU), and HU inhibition of Rr has previously been shown to inhibit HIV replication in vitro (Giacca et al., 1996). Furthermore, the addition of HU to nucleoside reverse transcriptase inhibitor therapy (NRTI) appears to enhance their antiviral activity both in vitro and in vivo.

Gallium is known to accumulate in certain tumors, inflamed tissue, and bone tissue by mechanisms that are largely unknown. Binding of gallium to transferring, particularly lactoferrin, is thought to be responsible for some of the transport of gallium in the body, and for the concentration of gallium in certain tumors and inflamed tissues. Radioactive ⁶⁷Ga citrate compositions are used in patients to diagnose certain malignancies and infections, including those in bone tissue. Non-radioactive gallium compositions, and compositions containing other Group IIIa elements, have been found effective in treating some tumors in animals and humans. Gallium is thought to be the most effective of these Group IIIa elements.

The art recognizes that gallium is useful for the treatment and prevention of many human and other mammalian diseases, including hypercalcemia, cancer, and certain degenerative or metabolic bone diseases such as osteoporosis and Paget's disease. It also has been used to treat infections, for example, those caused by Pseudomonas aeruginosa. U.S. Pat. No. 5,997,912. Gallium itself appears to be the active agent; the form in which the gallium is administered (e.g., as the nitrate, sulfate, or chloride) does not appear to affect its activity to any significant extent. Gallium nitrate (Ga) has been shown to be a potent inhibitor of ribonucleotide reductase. Interestingly, Ga was previously shown to inhibit avian retroviruses, although the mechanism of action was not elucidated. Waalkes (1974).

U.S. Pat. No. 5,525,598 describes gallium (III) compositions and their use in the treatment of cancers and viral infections. In one example, inhibition of HIV cell killing is described. Although the inventors claimed their activity, the results showed very poor therapeutic indices for gallium (III), ranging from 1.52 to 76.9, as compared with 1027 for AZT and 3680 for 3′-azido-2,3′-deoxythymidine.

### SUMMARY OF THE INVENTION

Thus, there is provided, in accordance with the present invention, a method of inhibiting human immunodeficiency virus (HIV) ribonucleotide reductase (Rr) in a subject infected with HIV comprising administering to said cell an amount of a gallium composition effective to inhibit Rr. HIV may be HIV-1 or HIV-2. The gallium composition may be gallium nitrate, or may be a gallium-hydroxypyrone complex. Alternatively, there is provided a method of inhibiting human immunodeficiency virus (HIV) replication in a subject infected with HIV comprising administering to said cell an amount of a gallium composition effective to inhibit HIV replication.

In another embodiment, there is provided a method of treating a human subject infected with human immunodeficiency virus (HIV) comprising administering to said subject an amount of a gallium composition effective to inhibit HIV replication. HIV may be HIV-1 or HIV-2. The gallium composition may be gallium nitrate, or may be a gallium-hydroxypyrone complex. The effective amount can be described as: achieving in vivo concentrations of about 1 to about 30 μM, or more specifically about 3 to about 20 μM. Alternatively, the effective amount is about 750 mg/m² given every two to three weeks, or about 100 to about 300 mg/m² per day. In one embodiment, the gallium composition is provided at levels sufficient to provide a blood plasma gallium concentration of 0.1 to 5.0 μg/ml. The gallium composition may be administered orally, for example, in the form of a tablet or a capsule. Alternatively, the gallium composition is administered intravenously.

In yet another embodiment, the method further comprises treating the subject with a second anti-viral agent in addition to the gallium composition, for example, a nucleoside analog that inhibits reverse transcriptase (NRTIs). Nucleoside analogs include dideoxyinosine, dideoxycytidine and 5-azidothymidine. Other anti-viral agents include protease inhibitors and non-nucleoside reverse transcriptase inhibitors (NNRTIs).

In still yet another embodiment, there is provided a method of reducing virus shed from a human subject infected with HIV comprising administering to said subject an amount of a gallium composition effective to inhibit HIV replication. The method may also provide for reduced virus burden in a human subject infected with HIV, inhibition of loss of T cells in a human subject infected with HIV, increase in T cell numbers, in a human subject infected with HIV, or inhibition of development of acquired immunodeficiency syndrome in a human subject infected with HIV.

In still a further embodiment, there is provided a therapeutic composition comprising (a) a gallium composition; and (b) a nucleoside inhibitor. The gallium composition may be gallium nitrate, or may be a gallium-hydroxypyrone complex. The NTRI may be one or more of the compounds selected from the group of didexoyinosine, dideoxycytidine and 5-azidothymidine. Also provided is a kit comprising, in suitable container means (a) a gallium composition; and (b) a nucleoside reverse transcriptase inhibitor.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Gallium Nitrate and Hydroxyurea Inhibition of HIV-1 Replication. PHA/IL-2 stimulated human PBMC's (1×10⁶), and MT-2 cells (1×10⁶) were grown and maintained in RPMI 1640 media containing 20% fetal calf sera, glutamine and antibiotics (Thelander & Reichard, 1979). Experiments were carried out in duplicate, and experiments repeated a minimum of three times. Gallium nitrate (Ga) was obtained from the National Cancer Institute. Hydroxyurea (HU) was obtained from Sigma Chemicals (St. Louis, Mo.). Ga and HU were added to PBMC's 24 hrs prior to infection, and cells were washed 16 hrs post-infection. Culture supernatants were obtained 4 and 7 days post-infection for p24 antigen measurement.

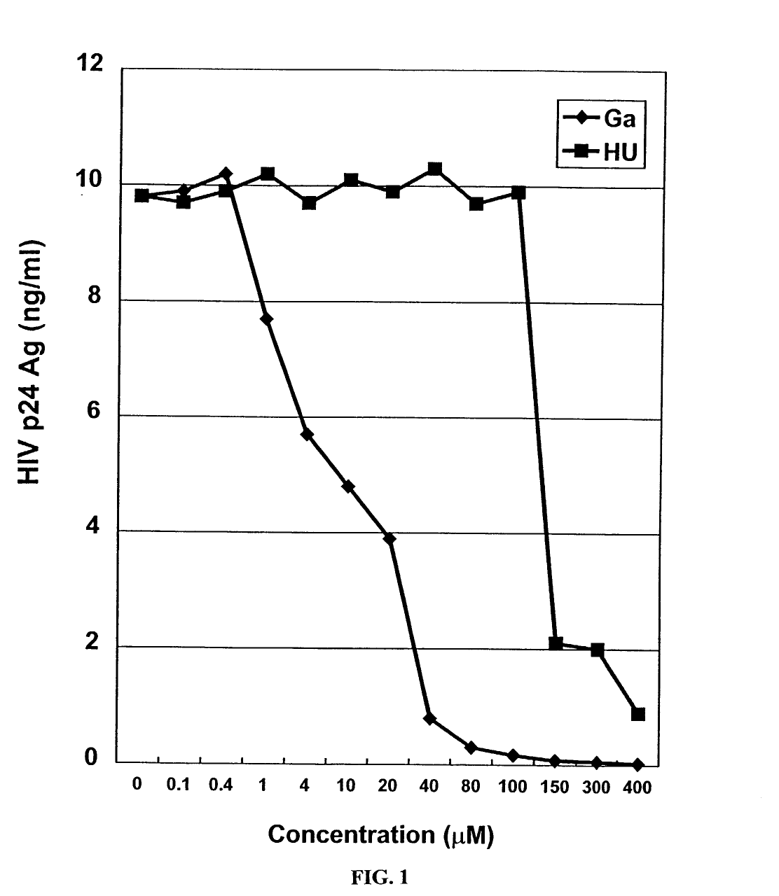

FIG. 2—Anti-HIV-1 Activity of Gallium Nitrate in Combination with Zidovudine and Didanosine. PHA/IL-2 stimulated human PBMC's (1×10⁶), and MT-2 cells (1×10⁶) were grown and maintained in RPMI 1640 media containing 20% fetal calf sera, glutamine and antibiotics (Thelander & Reichard, 1979). Experiments were carried out in duplicate, and experiments repeated a minimum of three times. Gallium nitrate (Ga) was obtained from the National Cancer Institute. Zidovudine (ZDV) and didanosine (ddI) were obtained from Sigma Chemicals (St. Louis, Mo.). Ga was added to PBMC's 24 hrs prior to infection, and cells were washed 16 hrs post-infection. Culture supernatants were obtained 4 and 7 days post-infection for p24 antigen measurement. Nucleoside reverse transcriptase inhibitors were added at the time of infection.

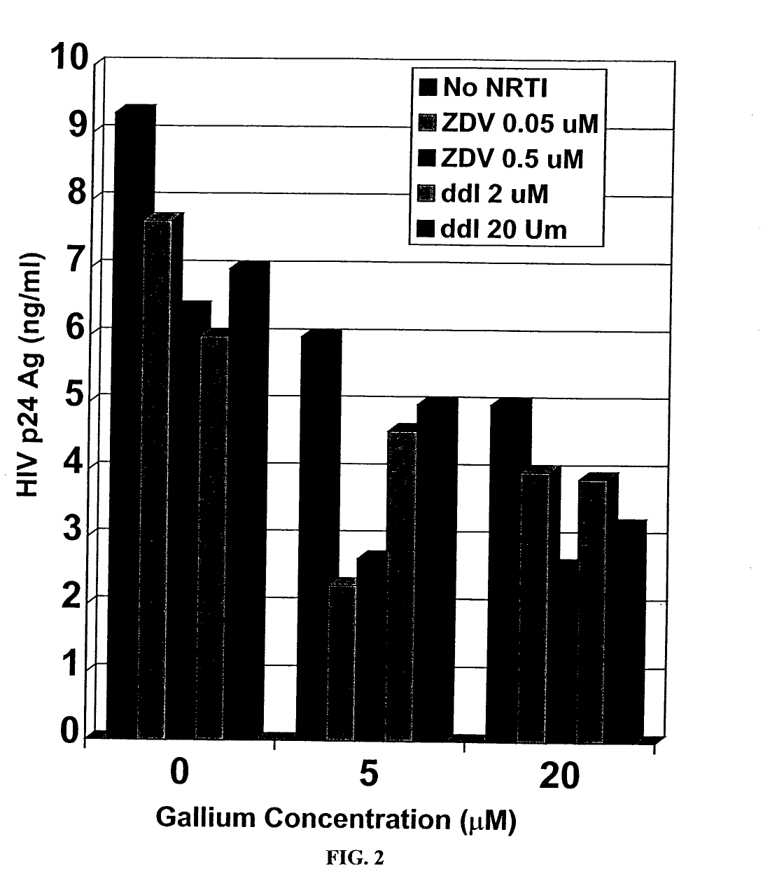

FIG. 3—Effects of Gallium Nitrate and Hydroxyurea on HIV-1 Induced Syncytia Formation. PHA/IL-2 stimulated human PBMC's (1×10⁶), and MT-2 cells (1—10⁶) were grown and maintained in RPMI 1640 media containing 20% fetal calf sera, glutamine and antibiotics (Thelander & Reichard, 1979). Experiments were carried out in duplicate, and experiments repeated a minimum of three times. Gallium nitrate (Ga) was obtained from the National Cancer Institute. Hydroxyurea (HU) was obtained from Sigma Chemicals (St. Louis, Mo.). Ga and HU were added to PBMC's 24 hrs prior to infection, and cells were washed 16 hrs post-infection. Cells were observed daily for syncytia induction.

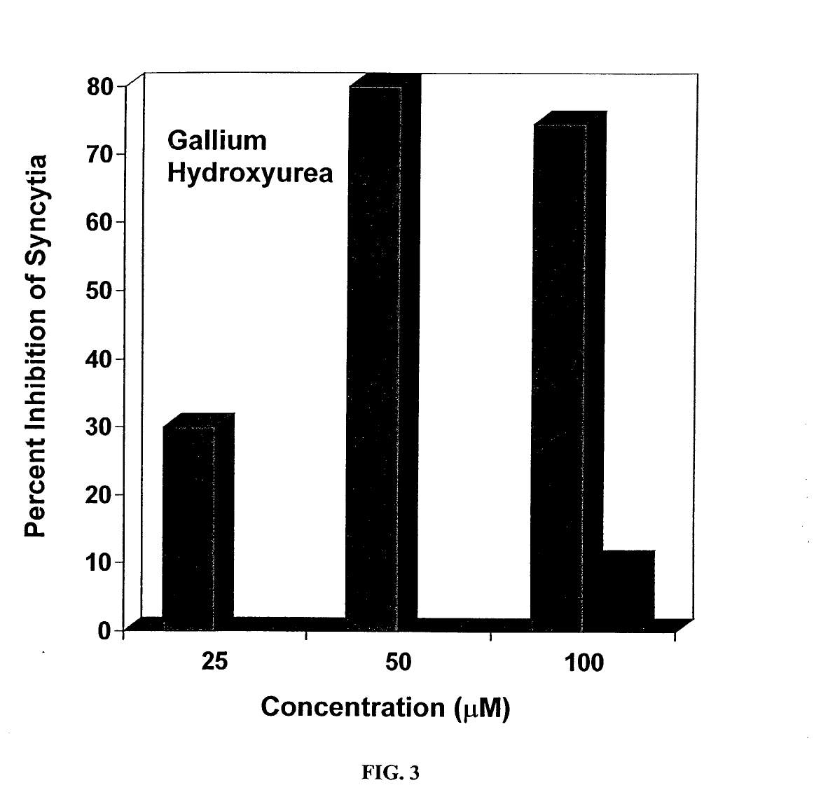

FIG. 4—Effects of Gallium Nitrate on PBMC Proliferation and Viability. Experiments were carried out in duplicate, and experiments repeated a minimum of three times. Gallium nitrate (Ga) was obtained from the National Cancer Institute. Cells were incubated in Ga at concentrations noted, and were stained at 48 hrs with Trypan blue and counted.

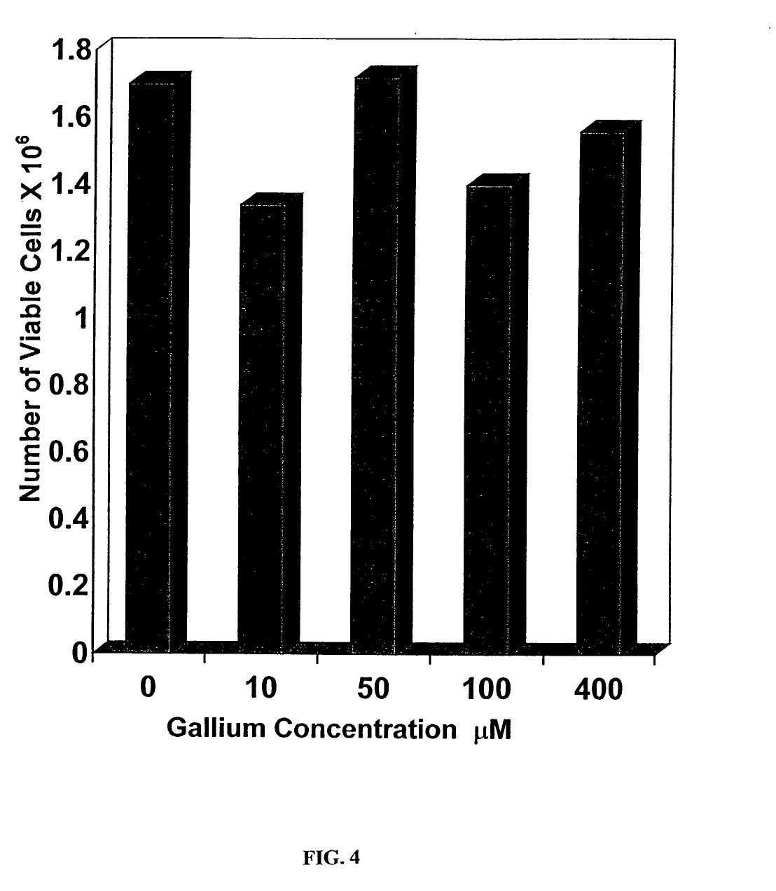

### DESCRIPTION OF ILLUSTRATIVE EMBODIMENTS

Human immunodeficiency virus (HIV) continues to present world wide health problems, with the number of infected individuals growing rapidly in both industrialized and third-world countries. In addition, healthcare costs for treating HIV infections, and the ensuring acquired immunodeficiency syndrome, measure in the billions of dollars. While some therapies have shown success in limiting the course of the disease, there remains a need for new treatments, both to reduce the side effects seen with current therapy, and to reduce the development of drug resistant viruses.

I. The Present Invention

Gallium (Ga), including gallium nitrate, is a potent ribonucleotide reductase inhibitor which was previously shown to inhibit avian retroviruses (Waalkes, 1974). Although the mechanism of its anti-retroviral activity was not elucidated, it is known that Ga inhibits cellular activation in a manner analogous to hydroxyurea (HU). U.S. Pat. No. 5,525,598 also reported anecdotal evidence that gallium (III) inhibits HIV-induced cell killing. Since Ga is administered to humans intravenously, and oral preparations currently are being developed, the inventors chose to evaluate Ga for potential anti-HIV activity, comparing it with HU.

Various concentrations of Ga or HU were added to PHA/IL2 stimulated PBMC's 24 hours prior to infection with HIV. Following infection, cells were washed and culture supernatants were obtained 4 and 7 days post-infection. Ga reproducibly inhibited HIV replication at concentrations which did not inhibit cellular proliferation or viability. Ga IC₅₀ ranged from 4 to 10 μM, which was approximately 15-fold lower than HU (120 μM) in the present culture system. Further, subinhibitory concentrations of zdv, ddI and ddC were rendered inhibitory by coadministration with Ga, indicate that Ga can potentiate the effects of these nucleoside analogs.

Thus, the inventors conclude that Ga is not only considerably more potent than HU in inhibiting HIV-1 replication in stimulated PBMC and MT-2 cell cultures, but that it can potentiate the effect of anti-HIV nucleoside RT inhibitors. Ga inhibits the same cellular target as HU, although it apparently does so by a different mechanism of action. Since the inhibitory concentration of Ga is achievable in humans, and the relative potency of Ga is greater than HU, Ga is a suitable agent for use, alone or in combination therapy, in treating HIV infections.

II. HIV

HIV is a single-stranded RNA virus. The provirus contains two long terminal repeats bounding a central region that encodes the three essential structural genes, gag, pol, and env. The gag gene encodes a polyprotein precursor that is cleaved by the viral protease during maturation. The pol gene encodes a precursor that contains protease-reverse transcriptase-endonuclease (in that order) functions. Env encodes a glycosylated polypeptide precursor (gp160) that is processed to form two membrane glycoproteins (g120 and gp41), the smaller of which is a transmembrane protein that serves as an anchor for the larger.

A number of genes encoding accessory proteins also have been identified: vif, a cytoplasmic/inner membrane protein involved in infectivity; tat, nuclear protein involved in transcriptional and post-translational activation; rev, nuclear protein involved in expression of structural proteins and modulation of transcription; nef, cytoplasmic protein; vpr; and vpu. The tat protein plays a major role in the virus lifecycle, activating transcription via the “TAR” sequence found in the LTR. Other sequences, including NFκB, GC boxes and TATA also play a role.

The life-cycle of HIV follows the general outline seen with other retroviruses. The cycle begins with binding to the cellular receptor—CD4. Studies suggest that entry is accomplished at the cell surface by fusion of viral and host membranes. Once inside the cell, reverse transcription of the viral genome occurs, followed by integration into the host cell genome. Viral proteins and DNA is then produced. Packaging of new genomes into viral particles and release follow.

III. Gallium Compositions and Dosages

Gallium compositions and uses are disclosed in U.S. Pat. Nos. 4,529,593, 4,596,710, 4,704,277, 5,747,482 and 5,883,088, each of which are specifically incorporated by reference.

U.S. Pat. No. 5,883,088 discloses pharmaceutical compositions that comprise gallium complexes of 3-hydroxy-4-pyrones. These compositions provide enhanced gallium bioavailability particularly when orally administered as compared to the gallium bioavailability achieved by use of pharmaceutical compositions containing gallium salts. Compositions included in this invention are useful in providing gallium to humans and other mammals for a wide variety of medical and veterinary applications, including the treatment, prevention, or diagnosis of hypercalcemia, certain cancers, certain disorders of calcium homeostasis, and certain bone diseases including osteoporosis, osteopenia, and Paget's disease. Related patents include U.S. Pat. Nos. 5,968,922; 5,981,518; 5,998,397; 6,048,851; and 6,087,354, also incorporated by reference.

U.S. Pat. No. 4,596,710 provides for the application of gallium chloride in the treatment of malignant tumors. Pharmaceutical compositions particularly intended for oral administration and containing from 100 to 500 mg of active ingredient per unit dose are described.

U.S. Pat. No. 4,529,593 teaches preventing or treating a disorder associated with accelerated loss of calcium from bone in a human individual by administering to the individual a pharmaceutically acceptable gallium compound. Of special importance among the disorders which may be thus prevented or treated are hypercalcemia, accelerated bone loss associated with osteopenia, osteoporosis, bone metastasis due to malignant tumors, and hyperparathyroidism. Gallium compounds may be administered by any and all routes. Although all biocompatible, soluble compounds of gallium may be used, gallium nitrate is said to be preferred, most preferably in a pharmaceutically acceptable carrier.

U.S. Pat. No. 5,747,482 invention is directed to the discovery that certain neutral gallium chelates, when used at a sufficient concentration, inhibit the proliferation of keratinocytes. Accordingly, such chelates can be used to treat disease conditions characterized by excessive proliferation of keratinocytes such as psoriasis. The reference suggests (a) that the active moiety is the chelated gallium complex rather than elemental gallium and/or unbound chelator and (b) that while neutral gallium chelates are capable of inhibiting keratinocyte proliferation, other gallium compounds, such as gallium nitrate, are not.

U.S. Pat. No. 4,704,277 describes gallium compounds that increase bone growth, decrease hydroxyapatite solubility, increase the size and/or the perfection of hydroxyapatite crystals in bone, and increase the tensile strength of bone. These compounds, when administered to patients who are suffering from diseases characterized by bone resorption impede the flow of bone calcium into the blood, and encourage the growth of new, normal bone tissue.

IV. Combination Therapies

The present inventors have determined that not only do Gallium compositions provide an effective therapy for HIV on their own, they also improve the efficacy of other traditional anti-viral compounds as well. In particular, the present inventors have shown Gallium nitrate potentiates the effects of NRTIs such as didexoyinosine, dideoxycytidine and azidothymidine. Other combination therapies are envisioned, such as combining Gallium with protease inhibitors or NNRTIs. This is important not only in the creation of more effective therapies, but in reducing the chance that drug-resistant viruses will develop.

To inhibit virus replication and thereby limit infection and T cell loss, using the methods and compositions of the present invention, one will treat a patient with a Gallium composition and a traditional antiviral therapeutic. This process may involve administration of both therapies at the same time, for example, by administration of a single composition or pharmacological formulation that includes both agents, or by administering to said patient two distinct compositions or formulations, at the same time.

Alternatively, the traditional therapy may precede or follow the present Gallium composition treatment by intervals ranging from minutes to weeks. It is also conceivable that more than one administration of either treatment will be desired. Various combinations may be employed, where the Gallium composition is “A” and the traditional therapeutic is “B”:

A/B/A B/A/B B/B/A A/A/B A/B/B B/A/A B/B/B/A B/B/A/B
A/A/B/B A/B/A/B A/B/B/A B/B/A/A B/A/B/A B/A/A/B
A/A/A/B B/A/A/A A/B/A/A A/A/B/A A/B/B/B B/A/B/B

In another aspect of combination therapies, it should be noted that one of the complications of AIDS is the increased frequency of tuberculosis. It has been observed that Gallium has an inhibitory effect on Mycobacterium tuberculosis distinct from its effects on HIV. In particular, it is believed that Gallium interferes with M. tuberculosis by competing with element iron (Fe) and/or interfering with Fe-dependent metabolic machinery, often in macrophages. See U.S. Ser. No. 08/707,248, filed Sep. 3, 1996.

V. Pharmaceutical Compositions and Routes of Administration

Where clinical applications are contemplated, it will be necessary to prepare pharmaceutical compositions of the compositions in a form appropriate for the intended application. Generally, this will entail preparing compositions that are essentially free of pyrogens, as well as other impurities that could be harmful to humans or animals.

One will generally desire to employ appropriate salts and buffers to render the compositions suitable for introduction into a patient. Aqueous compositions of the present invention comprise an effective amount of the therapeutic agent dissolved or dispersed in a pharmaceutically acceptable carrier or aqueous medium, and preferably encapsulated. The phrase “pharmaceutically or pharmacologically acceptable” refer to molecular entities and compositions that do not produce adverse, allergic, or other untoward reactions when administered to an animal or a human.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients, such as other anti-viral agents, can also be incorporated into the compositions.

Solutions of the active ingredients as free base or pharmacologically acceptable salts can be prepared in water suitably mixed with surfactant, such as hydroxypropylcellulose. Dispersions also can be prepared in glycerol, liquid polyethylene glycols, mixtures thereof and in oils. Under ordinary conditions of storage and use, these preparations contain a preservative to prevent growth of microorganisms. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

An effective amount of the composition is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

Also contemplated are combination compositions that contain two active ingredients. In particular, the present invention provides for compositions that contain gallium compositions and at least a second anti-viral drug, for example, a nucleoside analog or a protease inhibitor.

1. Parenteral Administration

The active compositions of the present invention may be formulated for parenteral administration, e.g., formulated for injection via the intravenous, intramuscular, subcutaneous, or even intraperitoneal routes. The preparation of an aqueous composition that contains a second agent(s) as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the particular methods of preparation are vacuum-drying and freeze-drying techniques which yield a powder of the active ingredient plus any additional desired ingredient from a previously sterile-filtered solution thereof.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

2. Other Routes of Administration

In addition to the compounds formulated for parenteral administration, oral formulations are provided. These compositions take the form of solutions, suspensions, tablets, pills, capsules, sustained release formulations or powders and include such typical excipients as, for example, pharmaceutical grades of mannitol, lactose, starch, magnesium stearate, sodium saccharine, cellulose, magnesium carbonate and the like. Similar compositions are provided or nasal, buccal, rectal, vaginal or topical administration.

VI. Kits

The present invention also provides therapeutic kits. Such kits will generally contain, in suitable container means, a pharmaceutically acceptable formulation of a gallium composition and at least one nucleoside or non-nucleoside reverse transcriptase inhibitor, or a protease inhibitor. The kits may also contain other pharmaceutically acceptable formulations, such as any other anti-viral compound in accordance with the invention.

The kits may have a single container means that contains both the gallium compound and the other agent, or the compositions may be found in separate containers. Certain kits of the present invention include gallium, packaged for use in combination with the co-administration of an NRTI, protease inhibitor or an NNRTI. In such kits, the gallium and the nucleoside analog may be pre-complexed, either in a molar equivalent combination, or with one component in excess of the other; or each of the gallium compound and the nucleoside analog maintained separately within distinct containers prior to administration to a patient.

When the components of the kit are provided in one or more liquid solutions, the liquid solution is an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container means.

The container means of the kit will generally include at least one vial, test tube, flask, bottle, syringe or other container means, into which the gallium, and other desired agent, may be placed and, preferably, suitably aliquoted. Where additional components are included, the kit will also generally contain a second vial or other container into which these are placed, enabling the administration of separated designed doses. The kits may also comprise a second/third container means for containing a sterile, pharmaceutically acceptable buffer or other diluent.

The kits may also contain a means by which to administer the gallium and other drug to an animal or patient, e.g., one or more needles or syringes, or even an eye dropper, pipette, or other such like apparatus, from which the formulation may be injected into the animal or applied to a diseased area of the body. The kits of the present invention will also typically include a means for containing the vials, or such like, and other component, in close confinement for commercial sale, such as, e.g., injection or blow-molded plastic containers into which the desired vials and other apparatus are placed and retained.

VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials & Methods

Virus Preparations:

HIV (Catalog# 1073, NIH AIDS Reference Program) was used in these studies. Virus stocks had been titered in appropriate cell lines as previously described (Cook et al., 1997); the HIV stock resulted in 4+ syncytia 48-72 hrs. post-infection. Viral replication was measured by determining the concentration of p24 antigen in culture supernatants (Organon-Teknika), and by visually observing a reduction in syncytia formation (Cook et al., 1997). In addition, a quantitative measurement of syncytia induction—inhibition was employed (described below).

Cells and Inhibitors:

PHA/IL-2 stimulated human PBMC's (1×10⁶), and MT-2 cells (1×10⁶) were grown and maintained in RPMI 1640 media containing 20% fetal calf sera, glutamine and antibiotics (Thelander & Reichard, 1979). Experiments were carried out in duplicate, and experiments repeated a minimum of three times. Gallium nitrate (Ga) was obtained from the National Cancer Institute. Hydroxyurea (HU), zidovudine (ZDV), and didanosine (ddI) were obtained from Sigma Chemicals (St. Louis, Mo.). Ga and HU were added to PBMC's 24 hrs prior to infection, and cells were washed 16 hrs post-infection. Cells were observed daily for syncytia induction, and culture supernatants were obtained 4 and 7 days post-infection for p24 antigen measurement. Nucleoside reverse transcriptase inhibitors were added at the time of infection.

Quantitative Syncytia Reduction Assay:

The inventors developed a fluorescence-labeling system to directly measure and quantitate HIV-1 induced syncytia formation. 10⁶ MT-2 cells were pelleted, and 50% were stained with 3 μM CMTMR dye (green), and the other half with 3 μM CMFDA dye (red) for 30 min at 37° C. in 5% CO₂. Cells were washed, then incubated for 45 min in fresh media prior to infecting the cells with HIV-1. The infected, differentially stained cells were mixed, incubated for 48 hrs, fixed in 4% paraformaldehyde, and analyzed by confocal microscopy (Zeiss) for double-staining (indicating HIV-induced fusion). Quantitation was accomplished by measuring the number and size of dually stained cells per field, and by calculating cell volume equivalents. For experiments with HIV-1 inhibitors, the inhibitor was added two hours post-infection.

All of the Compositions and/or Methods disclosed and claimed herein can be made and executed without undue experimentation in light of the present disclosure. While the compositions and methods of this invention have been described in terms of preferred embodiments, it will be apparent to those of skill in the art that variations may be applied to the Compositions and/or Methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Cook et al., “Effects of a single ethanol exposure on HIV replication in human lymphocytes.” J. Invest. Med. 45:265, 1997.

Giacca et al., “Synergistic antiviral action of ribonucleotide reductase inhibitors and 3′-azido-3′deoxythymidine on HIV type 1 infection in vitro.” AIDS 12:677, 1996.

Thelander & Reichard, “Reduction of ribonucleotides.” Annu. Rev. Biochem. 48:133, 1979.

Waalkes, Cancer Res. 34:385, 1974.

U.S. Pat. No. 4,529,593

U.S. Pat. No. 5,596,710

U.S. Pat. No. 4,704,277

U.S. Pat. No. 5,525,598

U.S. Pat. No. 5,747,482

U.S. Pat. No. 5,883,088

U.S. Pat. No. 5,968,922

U.S. Pat. No. 5,981,518

U.S. Pat. No. 5,997,912

U.S. Pat. No. 5,998,397

U.S. Pat. No. 6,048,851

U.S. Pat. No. 6,087,354

WO 93/02087

What is claimed is:

1. A method of inhibiting human immunodeficiency virus (HIV) ribonucleotide reductase (Rr) in a subject infected with HIV comprising administering to said subject an amount of a gallium composition effective to inhibit Rr.

2. The method of claim 1, wherein HIV is HIV-1.

3. The method of claim 1, wherein HIV is HIV-2.

4. The method of claim 1, wherein HIV has infected a T-cell.

5. The method of claim 1, wherein said gallium composition is gallium nitrate.

6. The method of claim 1, wherein said gallium composition is a gallium-hydroxypyrone complex.

7. A method of inhibiting human immunodeficiency virus (HIV) replication in a subject infected with HIV comprising administering to said subject an amount of a gallium composition effective to inhibit HIV replication.

8. The method of claim 7, wherein HIV is HIV-1.

9. The method of claim 7, wherein HIV is HIV-2.

10. The method of claim 1, wherein HIV has infected a T-cell.

11. A method of treating a human subject infected with human immunodeficiency virus (HIV) comprising administering to said subject an amount of a gallium composition effective to inhibit HIV replication.

12. The method of claim 11, wherein HIV is HIV-1.

13. The method of claim 11, wherein HIV is HIV-2.

14. The method of claim 11, wherein said gallium composition is gallium nitrate.

15. The method of claim 11, wherein said gallium composition is a gallium-hydroxypyrone complex.

16. The method of claim 11, wherein said effective amount achieves in vivo concentrations of about 1 to about 30 μM.

17. The method of claim 16, wherein said effective amount is about 3 to about 20 μM.

18. The method of claim 11, wherein said effective amount is about 750 mg/m² given every two to three weeks.

19. The method of claim 11, wherein said effective amount is about 100 to about 300 mg/m² per day.

20. The method of claim 11, wherein said effective amount is given in a unit dose of about 200 mg to about 1000 mg.

21. The method of claim 11, wherein said gallium composition is administered orally.

22. The method of claim 21, wherein said gallium composition is in the form of a tablet.

23. The method of claim 21, wherein said gallium composition is in the form of a capsule.

24. The method of claim 11, wherein said gallium composition is administered intravenously.

25. The method of claim 11, wherein said gallium composition is sufficient to provide a blood plasma gallium concentration of 0.1 to 5.0 μg/ml.

26. The method of claim 11, further comprising treating said subject with a second anti-viral agent.

27. The method of 26, wherein said second anti-viral agent is a nucleoside reverse transcriptase inhibitor (NRTI).

28. The method of claim 26, wherein said NRTI is didexoyinosine.

29. The method of claim 26, wherein said NRTI is dideoxycytidine.

30. The method of claim 26, wherein said NRTI is 5-azidothymidine.

31. A method of reducing virus shed from a human subject infected with human immunodeficiency virus (HIV) comprising administering to said subject an amount of a gallium composition effective to inhibit HIV replication.

32. A method of reducing virus burden in a human subject infected with human immunodeficiency virus (HIV) comprising administering to said subject an amount of a gallium composition effective to inhibit HIV replication.

33. A method of inhibiting loss of T cells in a human subject infected with human immunodeficiency virus (HIV) comprising administering to said subject an amount of a gallium composition effective to inhibit HIV replication.

34. The method of claim 33, wherein the number of T cells in said subject increases following treatment with said gallium composition.

35. A method of inhibiting development of acquired immunodeficiency syndrome in a human subject infected with human immunodeficiency virus (HIV) comprising administering to said subject an amount of a gallium composition effective to inhibit HIV replication.

36. A therapeutic composition comprising:
(a) a gallium composition; and
(b) a nucleoside inhibitor.

37. The composition of claim 36, wherein said gallium composition is gallium nitrate.

38. The composition of claim 36, wherein said gallium composition is a gallium-hydroxypyrone complex.

39. The composition of claim 36, wherein the nucleoside inhibitor is one or more of the compounds selected from the group of dideoxyinosine, dideoxycytidine and 5-azidothymidine.

40. A kit comprising, in suitable container means:
(a) a gallium composition; and
(b) a nucleoside reverse transcriptase inhibitor.
